(19) United States
(12) Patent Application Publication
(10) Pub. No.: US 2020/0054649 A1
(43) Pub. Date: Feb. 20, 2020

(54) SKELETAL MUSCLE HYPERTROPHY INDUCERS

(71) Applicant: CYTOO, GRENOBLE (FR)

(72) Inventors: PAULINE POYDENOT, SAINT MARTIN D'HERES (FR); JORIS MICHAUD, LAUSANNE (CH); MÉLANIE FLAENDER, GRENOBLE (FR); EVE DUCHEMIN-PELLETIER, VIZILLE (FR); LUC SELIG, CHARENTON LE PONT (FR)

(21) Appl. No.: 16/461,088

(22) PCT Filed: Nov. 17, 2017
(86) PCT No.: PCT/EP2017/079667

§ 371 Date: May 15, 2019

(30) Foreign Application Priority Data
Nov. 17, 2016 (EP) 16306507.1

(51) Int. Cl. A61K 31/57; A23K 20/184; A61P 21/06

(52) U.S. Cl. CPC A61K 31/57; A61P 21/06; A23K 20/184

(57) ABSTRACT

The present invention relates to skeletal muscle hypertrophy inducers as well as their uses to promote skeletal muscle regeneration, to prevent skeletal muscle atrophy, or in the treatment or prevention of a disease or injury resulting in loss of skeletal muscle tissue and/or muscle weakness. It further relates to the non-therapeutic use of such compounds to increase muscle mass, muscle strength and/or muscle performance in a subject.

### FIELD OF THE INVENTION

The present invention relates to therapeutic strategies to induce skeletal muscle hypertrophy, prevent atrophy or treat or prevent diseases or injuries resulting in loss of skeletal muscle tissue and/or muscle weakness. It also relates to a non-therapeutic use of skeletal muscle hypertrophy inducers.

### BACKGROUND OF THE INVENTION

Muscle wasting and weakness may result from a large panel of disease states and conditions including metabolic diseases, neurologic diseases, muscle diseases, acute or chronic illness (cachexia), aging, inactivity, food starvation and even poisoning. During the last 15 years, extensive research has led to a better understanding of the signalling pathways implicated in the loss of muscle mass. However, to date, the offer of therapeutic strategies directly targeting the muscle remains poor.

Muscle loss may occur, in particular, with aging and is a component of the frailty syndrome. Named “sarcopenia”, this degenerative loss results in direct muscle atrophy and carries an increased risk for poor health outcomes including falls, incident disability, hospitalization, and mortality. With a growing older population, sarcopenia is an ever increasing global health concern and there has been great interest in developing approaches to counteract the effects of sarcopenia, and thereby reduce the age-related decline and disability. Potential interventions for sarcopenia may include physical activity and nutritional supplementation but, to date, pharmacological interventions have shown limited efficacy.

Muscle weakness can also directly result from neuromuscular disorders such as myopathies, neuromuscular junction diseases or motor neuron diseases.

Myopathies are neuromuscular disorders in which the primary symptom is muscle weakness due to dysfunction of skeletal muscle fibres. Myopathies can be inherited or acquired and include, for example, muscular dystrophies, metabolic myopathies such as mitochondrial myopathies or drug-induced myopathies, and autoimmune myopathies such as dermatomyositis, polymyositis or inclusion body myositis.

Among myopathies, muscular dystrophies represent a large group causing a progressive degeneration of myofibers and resulting in a loss of muscle mass. Mutations in over 30 genes causing muscular dystrophies have been identified. Duchenne Muscular Dystrophy (DMD) is the most common form of muscular dystrophy with an occurrence rate of about one in 3,500 males worldwide.

Treatments for neuromuscular disorders depend on the disease and specific causes, however, to date, there is no specific treatment to stop or reverse any form of muscular dystrophy. Exercise and nutritional interventions have merit for slowing the rate of muscle atrophy in some muscle wasting conditions, but in most cases they cannot halt the wasting process.

Therefore, there is a strong need for new therapeutic options that can efficiently attenuate muscle atrophy, promote muscle growth, increase muscle mass and ultimately improve the quality of life for patients.

### SUMMARY OF THE INVENTION

The object of the present invention is to provide new therapeutic strategies to induce skeletal muscle hypertrophy, or prevent muscular atrophy, promote skeletal muscle regeneration, and treat or prevent skeletal muscle wasting.

In a first aspect, the invention relates to a compound of formula (I)

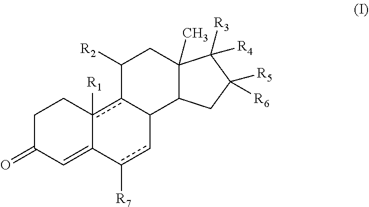

wherein

R₁ is hydrogen or a C₁-C₃ alkyl, or is absent;

R₂ is selected from the group consisting of hydrogen, a hydroxyl group, a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;

R₃ is a hydroxyl group, —C(═O)R₈ with R₈ being a C₁-C₃ alkyl optionally substituted by a hydroxyl group, or —O—C(═O)R₁₁ with R₁₁ being a C₁-C₆ alkyl group optionally substituted by a carboxyl group;

R₄ is hydrogen, an acetoxy group or a C₁-C₆ alkyl, C₂-C₆ alkenyl or C₂-C₆ alkynyl group optionally substituted by a hydroxyl group or a halogen; or

R₃ and R₄ taken together form a tetrahydrofuran group optionally substituted by a methylene group;

R₅ and R₆ are hydrogen or taken together form a methylene group;

R₇ is hydrogen or methyl

a and b respectively denote, independently from each other, a single bond or a double bond, with the proviso that R₁ is absent when a is a double bond;

with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group,

or any pharmaceutically acceptable diastereoisomer, salt, hydrate, solvate or prodrug thereof,

for use as skeletal muscle hypertrophy inducer, preferably in a subject suffering from a disease or disorder resulting in loss of skeletal muscle tissue and/or muscle weakness.

Preferably, formula (I) is

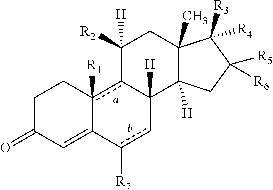

The invention also relates to a compound of formula (I)

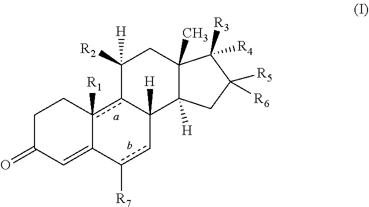

wherein

R₁ is hydrogen or a C₁-C₃ alkyl, or is absent;

R₂ is selected from the group consisting of hydrogen, a hydroxyl group, a 4-dimethylamino-phenyl group, a 4-methylamino-phenyl group and an aminophenyl group;

R₃ is a hydroxyl group or —C(═O)R₈ with R₈ being a C₁-C₃ alkyl or —CH₂OH;

R₄ is hydrogen, an acetoxy group or a propynyl group;

R₅ and R₆ are hydrogen or taken together form a methylene group;

R₇ is hydrogen or methyl

a and b respectively denote, independently from each other, a single bond or a double bond, with the proviso that R₁ is absent when a is a double bond;

with the provisos that

(i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH, and (iii) R₄ is an acetoxy group or a propynyl group when R₃ is a hydroxyl group,

or any pharmaceutically acceptable salt, hydrate, solvate or prodrug thereof,

for use as skeletal muscle hypertrophy inducer, preferably in a subject suffering from a disease or disorder resulting in loss of skeletal muscle tissue and/or muscle weakness. It also relates to a compound of formula (I) as defined above, or any pharmaceutically acceptable salt, hydrate, solvate or prodrug thereof, for use to promote skeletal muscle regeneration and/or prevent skeletal muscle atrophy, preferably in a subject suffering from a disease or disorder resulting in loss of skeletal muscle tissue and/or muscle weakness.

It further relates to a compound of formula (I) as defined above, or any pharmaceutically acceptable salt, hydrate, solvate or prodrug thereof, for use in the treatment or prevention of a disease or disorder resulting in loss of skeletal muscle tissue and/or muscle weakness.

In some embodiments, R₁ is hydrogen or methyl, or is absent; R₂ is selected from the group consisting of hydrogen, a hydroxyl group and a 4-dimethylamino-phenyl group; R₃ is a hydroxyl group or —C(═O)R₈ with R₈ being methyl or —CH₂OH; R₄ is hydrogen, an acetoxy group or a 1-propynyl group; and R₇ is hydrogen or methyl.

In particular, R₁ may be hydrogen or methyl, preferably methyl, R₂ may be hydrogen or a hydroxyl group, R₃ may be —C(═O)R₈ with R₈ being methyl or —CH₂OH, and/or R₄ may be hydrogen or an acetoxy group, preferably acetoxy group.

In some embodiments,

R₁ is absent;

R₂ is a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;

R₃ is a hydroxyl group or —O—C(═O)R₁₁ with R₁₁ being a C₁-C₆ alkyl group optionally substituted by a carboxyl group;

R₄ is a C₁-C₆ alkyl, C₂-C₆ alkenyl or C₂-C₆ alkynyl group optionally substituted by a hydroxyl group or a halogen; or

R₃ and R₄ taken together form tetrahydrofuran group optionally substituted by a methylene group;

R₅ and R₆ are hydrogen; and

R₇ is hydrogen.

Preferably, R₂ is a phenyl group substituted, preferably in para position, by a dimethylamino group, a methylamino group, an amino group, a thiomethyl group, a dimethylamino N-oxide group, or —C(═O)R₁₀ with R₁₀ being a methyl. More preferably, R₂ is a phenyl group substituted, preferably in para position, by a dimethylamino group, a methylamino group or an amino group.

Preferably, R₃ is a hydroxyl group or —O—C(═O)R₁₁ with R₁₁ being an ethyl group optionally substituted by a carboxyl group. More preferably, R₃ is a hydroxyl group.

Preferably, R₄ is a C₂-C₃ alkyl group, C₂-C₃ alkenyl or C₂-C₃ alkynyl group optionally substituted by a hydroxyl group or a halogen, preferably chlorine. More preferably, R₄ is a propynyl group optionally substituted by a hydroxyl group.

R₃ and R₄ taken together may also form tetrahydrofuran group substituted by a methylene group.

In particular, the compound of formula I may be selected from the group consisting of

| Name | Formula |
|---|---|
| Mifepristone | 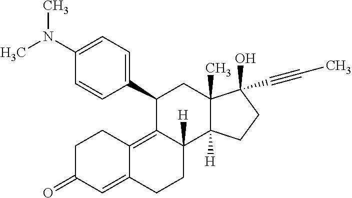 |
| Corticosterone | 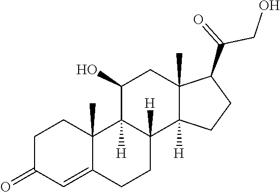 |
| Progesterone | 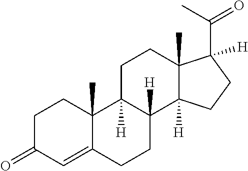 |
| Melengestrol acetate | 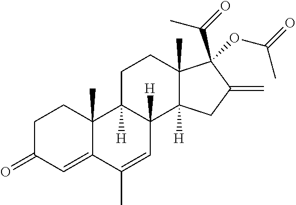 |
| Megestrol acetate | 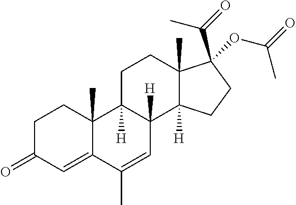 |
| Nestorone | 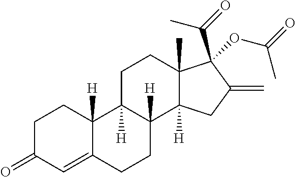 |
| RU42633 | 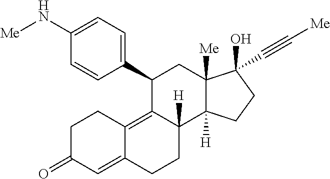 |
| RU42848 | 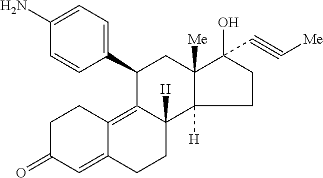 |
| RU42698 | 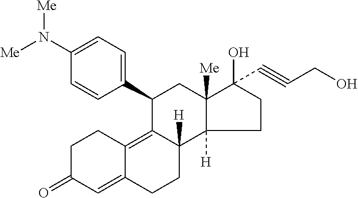 |
| Lilopristone | 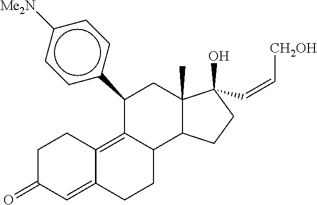 |
| Onapristone | 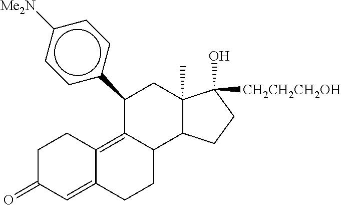 |

| | |
|---|---|
| ORG 31710 | 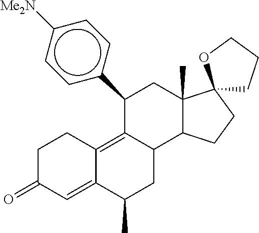 |
| ORG 33628 | 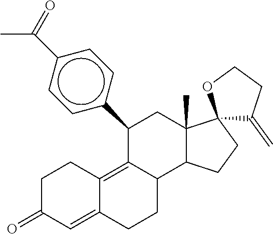 |
| RU 46556 | 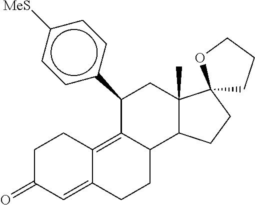 |
| RU 39973 | 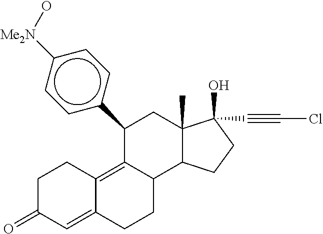 |
| RU 52562 | 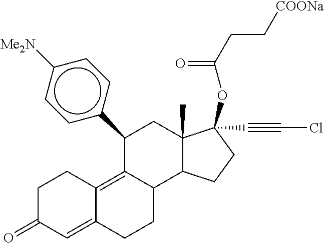 |
| Aglepristone | 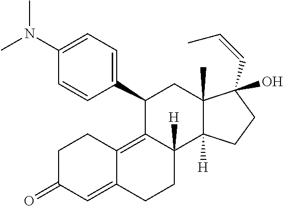 |

and any pharmaceutically acceptable diastereoisomer, salt, hydrate, solvate or prodrug thereof.

The compound may also be selected from the group consisting of corticosterone, progesterone, melengestrol acetate, megestrol acetate, nestorone and mifepristone, more preferably from corticosterone, nestorone and mifepristone, even more preferably may be mifepristone or nestorone.

Preferably, the compound of formula (I) is selected from the group consisting of mifepristone, and metabolites and analogues thereof, said metabolites being preferably selected from RU42633, RU42848 and RU42698 and said analogues being preferably selected from lilopristone, onapristone, aglepristone, ORG 31710, ORG 33628, RU 46556, RU 39973 and RU 52562. In particular, it may be selected from the group consisting of RU42633, RU42848 and RU42698, or any pharmaceutically acceptable salt, hydrate, solvate or prodrug thereof, or from the group consisting of lilopristone, onapristone, aglepristone, ORG 31710, ORG 33628, RU 46556, RU 39973 and RU 52562, or any pharmaceutically acceptable salt, hydrate, solvate or prodrug thereof.

The compound of formula I may be selected from the group consisting of mifepristone, RU42633, RU42848 and RU42968, preferably from the group consisting of mifepristone, RU42633 and RU42848, and more preferably is mifepristone.

The disease or disorder resulting in loss of skeletal muscle tissue and/or muscle weakness, may be selected from neuromuscular diseases, cachexia, sarcopenia, muscle disuse atrophy, atrophy induced by anorexia food starvation, and muscle injuries including acute muscular injury or muscle overuse injury, preferably selected from neuromuscular diseases, cachexia and sarcopenia, more preferably is sarcopenia or cachexia, and even more preferably is sarcopenia.

In another aspect, the present invention also relates to a product containing a compound of formula (I) as defined above, and a compound inducing skeletal muscular atrophy, as a combined preparation for simultaneous, separate or sequential use.

Preferably, the compound inducing skeletal muscular atrophy is a therapeutic agent, more preferably selected from the group consisting of corticosteroids, colchicine, chloroquine, hydroxychloroquine, D-penicillamine, antibiotics, betablockers, amiodarone, cimetidine, zidovudine, vincristine, clofibrate, statins, fibrates, cyclosporine, L-tryptophan, drugs causing hypokalaemia, lipid lowering agents, and therapeutic agents administered by intramuscular route such as vaccines, and even more preferably is a lipid lowering agent, such as statins and fibrates.

In another aspect, the present invention also relates to a non-therapeutic use of a compound of formula (I) as defined above, to increase muscle mass, muscle strength and/or muscle performance in a subject, and in particular to increase skeletal muscle mass, skeletal muscle strength and/or skeletal muscle performance in a subject.

The present invention also relates to the use, preferably the non-therapeutic use, of a compound of formula (I) as defined above, to prevent loss of skeletal muscle mass in a subject, or as ingredient or additive for animal feed composition.

The present invention further relates to a method of improving livestock performance comprising providing to said livestock a compound of formula (I) as defined above, preferably a feed composition, ingredient, additive, or dietary supplement comprising a compound of formula (I) as defined above.

### DETAILED DESCRIPTION OF THE INVENTION

Thanks to their solid knowledge on micropattern technology and a proprietary physiological human skeletal muscle model (MyoScreen™, CYTOO) allowing fully maturation of human primary myoblasts and providing myotubes with a high level of striation, high fusion index with aligned nuclei and low morphological variability, the inventors identified compounds exhibiting skeletal muscle hypertrophy activity. They showed that these hypertrophic compounds not only increase myotube differentiation and size from myoblasts, but are also able to prevent muscular atrophy.

Accordingly, in a first aspect, the present invention relates to the use of such compounds as skeletal muscle hypertrophy inducers, to promote skeletal muscle regeneration, to prevent skeletal muscle atrophy, or in the treatment or prevention of a disease or injury resulting in loss of skeletal muscle tissue and/or muscle weakness.

The present invention thus relates to a compound of formula (I)

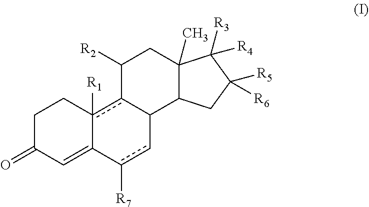

wherein

R₁ is hydrogen or a C₁-C₃ alkyl, or is absent;

R₂ is selected from the group consisting of hydrogen, a hydroxyl group, a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;

R₃ is a hydroxyl group, —C(═O)R₈ with R₈ being a C₁-C₃ alkyl optionally substituted by a hydroxyl group, or —O—C(═O)R₁₁ with R₁₁ being a C₁-C₆ alkyl group optionally substituted by a carboxyl group;

R₄ is hydrogen, an acetoxy group or a C₁-C₆ alkyl, C₂-C₆ alkenyl or C₂-C₆ alkynyl group optionally substituted by a hydroxyl group or a halogen; or

R₃ and R₄ taken together form a tetrahydrofuran group optionally substituted by a methylene group;

R₅ and R₆ are hydrogen or taken together form a methylene group;

R₇ is hydrogen or methyl

a and b respectively denote, independently from each other, a single bond or a double bond, with the proviso that R₁ is absent when a is a double bond;

with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group,

or any pharmaceutically acceptable diastereoisomer, salt, hydrate, solvate or prodrug thereof,

for use as skeletal muscle hypertrophy inducer.

Formula (I) encompasses all diastereoisomers of compounds defined above and in preferred embodiments, formula (I) is

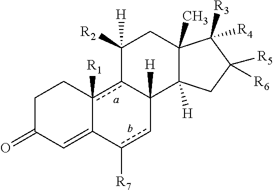

In particular embodiments, the compound is a compound of formula (I)

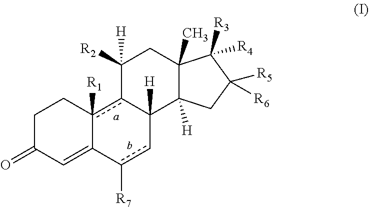

wherein

R₁ is hydrogen or a C₁-C₃ alkyl, or is absent;

R₂ is selected from the group consisting of hydrogen, a hydroxyl group, a 4-dimethylamino-phenyl group, a 4-methylamino-phenyl group and an aminophenyl group;

R₃ is a hydroxyl group or —C(═O)R₈ with R₈ being a C₁-C₃ alkyl or —CH₂OH;

R₄ is hydrogen, an acetoxy group or a propynyl group;

R₅ and R₆ are hydrogen or taken together form a methylene group;

R₇ is hydrogen or methyl;

a and b respectively denote, independently from each other, a single bond or a double bond,

with the proviso that R₁ is absent when a is a double bond; and with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group,

or any pharmaceutically acceptable salt, hydrate, solvate or prodrug thereof,

for use as skeletal muscle hypertrophy inducer.

As used herein, the term “alkyl” refers to a univalent radical containing only carbon and hydrogen atoms arranged in a chain. (C₁-C₃)-alkyl groups include methyl, ethyl, propyl, or isopropyl. Preferably, the (C₁-C₃)-alkyl group is methyl of ethyl, more preferably methyl. (C₁-C₆)-alkyl groups include methyl, ethyl, propyl, isopropyl, butyl, isobutyl, tert-butyl, pentyl or hexyl. Preferably, the (C₁-C₆)-alkyl group is methyl, ethyl, propyl or isopropyl.

As used herein, the term “alkenyl” refers to an unsaturated, linear or branched aliphatic group comprising at least one carbon-carbon double bound. The term “(C₂-C₆)alkenyl” more specifically means ethenyl, propenyl, isopropenyl, butenyl, isobutenyl, pentenyl, or hexenyl. Preferably, the (C₂-C₆)alkenyl group is ethenyl, propenyl or isopropenyl.

The term “alkynyl” refers to an unsaturated, linear or branched aliphatic group comprising at least one carbon-carbon triple bound. The term “(C₂-C₆)alkynyl” more specifically means ethynyl, propynyl, butynyl, pentynyl, isopentynyl, or hexynyl. Preferably, the (C₂-C₆)alkynyl group is ethynyl or propynyl, more preferably 1-propynyl group.

The term “thioalkyl” corresponds to the alkyl group as above defined bounded to the molecule by a —S— (thioether) bound. (C₁-C₃)thioalkyl group includes thio-methyl, thio-ethyl, thio-propyl. Preferably, the (C₁-C₃)thioalkyl is thio-methyl. As used herein, the term “dimethylamino N-oxide group” refers to —N(O)—(CH₃)₂.

The term “halogen” corresponds to a fluorine, chlorine, bromine, or iodine atom, preferably a chlorine.

In an embodiment, the compound of formula (I) has one or several of the following features:

a) R₁ is hydrogen or methyl, or is absent,

b) R₂ is selected from the group consisting of hydrogen, a hydroxyl group and a 4-dimethylamino-phenyl group,

c) R₃ is a hydroxyl group or —C(═O)R₈ with R₈ being methyl or —CH₂OH;

d) R₄ is hydrogen, an acetoxy group or a 1-propynyl group; and

e) R₇ is hydrogen or methyl.

In another embodiment, the compound of formula (I) has one or several of the following features:

a) R₁ is hydrogen or methyl, preferably methyl,

b) R₂ is selected from the group consisting of hydrogen and a hydroxyl group,

c) R₃ is —C(═O)R₈ with R₈ being methyl or —CH₂OH;

d) R₄ is hydrogen or an acetoxy group, preferably an acetoxy group; and

e) R₇ is hydrogen or methyl.

In particular, the compound of formula (I) may meet one feature, two features [for instance a) and b); a) and c); a) and d); a) and e); b) and c); b) and d); b) and e); c) and d); c) and e); d) and e)], three features [for instance a), b) and c); a), b) and d); a), b) and e); a), c) and d); a), c) and e); a), d) and e); b), c) and d); b), c) and e); c), d) and e)], four features [a), b), c) and d); a), b), c) and e); a), b), d) and e); a), c), d) and e); b), c), d) and e)], or five features [i.e. a), b), c), d) and e)] as described above.

In an embodiment,

R₁ is hydrogen or methyl, or is absent;

R₂ is selected from the group consisting of hydrogen, a hydroxyl group and a 4-dimethylamino-phenyl group;

R₃ is a hydroxyl group or —C(═O)R₈ with R₈ being methyl or —CH₂OH;

R₄ is hydrogen, an acetoxy group or a 1-propynyl group; and

R₇ is hydrogen or methyl.

In another embodiment,

R₁ is hydrogen or a C₁-C₃ alkyl, preferably methyl;

R₂ is selected from the group consisting of hydrogen and a hydroxyl group;

R₃ is —C(═O)R₈ with R₈ being a C₁-C₃ alkyl, preferably methyl, or —CH₂OH;

R₄ is hydrogen or an acetoxy group;

R₅ and R₆ are hydrogen or taken together form a methylene group; and

R₇ is hydrogen or methyl.

In another embodiment,

R₁ is hydrogen or a C₁-C₃ alkyl, preferably methyl;

R₂ is hydrogen;

R₃ is —C(═O)R₈ with R₈ being a C₁-C₃ alkyl, preferably methyl;

R₄ is hydrogen or an acetoxy group;

R₅ and R₆ are hydrogen or taken together form a methylene group; and

R₇ is hydrogen or methyl.

In another embodiment,

R₁ is C₁-C₃ alkyl, preferably methyl;

R₂ is hydrogen;

R₃ is —C(═O)R₈ with R₈ being a C₁-C₃ alkyl, preferably methyl;

R₄ is hydrogen or an acetoxy group;

R₅ and R₆ are hydrogen or taken together form a methylene group; and

R₇ is hydrogen or methyl.

In another embodiment,

R₁ is C₁-C₃ alkyl, preferably methyl;

R₂ is hydrogen;

R₃ is —C(═O)R₈ with R₈ being a C₁-C₃ alkyl, preferably methyl;

R₄ is an acetoxy group;

R₅ and R₆ are hydrogen or taken together form a methylene group; and

R₇ is methyl.

In another embodiment,

R₁ is C₁-C₃ alkyl, preferably methyl;

R₂ is hydrogen;

R₃ is —C(═O)R₈ with R₈ being a C₁-C₃ alkyl, preferably methyl;

R₄ is hydrogen or an acetoxy group;

R₅ and R₆ are hydrogen; and

R₇ is methyl or hydrogen.

In a further embodiment,

R₁ is a C₁-C₃ alkyl, preferably methyl, or is absent;

R₂ is selected from the group consisting of hydrogen, a hydroxyl group, a 4-dimethylamino-phenyl group, a 4-methylamino-phenyl group and an aminophenyl group, preferably from hydrogen, a hydroxyl group and a 4-dimethylamino-phenyl group;

R₃ is a hydroxyl group or —C(═O)R₈ with R₈ being a C₁-C₃ alkyl, preferably methyl, or —CH₂OH;

R₄ is hydrogen, an acetoxy group or a propynyl group, preferably a 1-propynyl group;

R₅ and R₆ are hydrogen or taken together form a methylene group; and

R₇ is hydrogen or methyl.

In another embodiment,

R₁ is hydrogen or a C₁-C₃ alkyl, preferably methyl, or is absent;

R₂ is selected from the group consisting of hydrogen, a hydroxyl group, a 4-dimethylamino-phenyl group, a 4-methylamino-phenyl group and an aminophenyl group, preferably from hydrogen, a hydroxyl group and a 4-dimethylamino-phenyl group;

R₃ is a hydroxyl group or —C(═O)R₈ with R₈ being a C₁-C₃ alkyl, preferably methyl, or —CH₂OH;

R₄ is hydrogen or a propynyl group, preferably a 1-propynyl group;

R₅ and R₆ are hydrogen or taken together form a methylene group; and

R₇ is hydrogen or methyl, preferably is hydrogen.

In another embodiment,

R₁ is hydrogen or a C₁-C₃ alkyl, preferably methyl, or is absent;

R₂ is selected from the group consisting of hydrogen, a hydroxyl group, a 4-dimethylamino-phenyl group, a 4-methylamino-phenyl group and an aminophenyl group, preferably from hydrogen, a hydroxyl group and a 4-dimethylamino-phenyl group;

R₃ is a hydroxyl group or —C(═O)R₈ with R₈ being a C₁-C₃ alkyl, preferably methyl, or —CH₂OH;

R₄ is hydrogen, an acetoxy group or a propynyl group, preferably a 1-propynyl group;

R₅ and R₆ are hydrogen or taken together form a methylene group; and

R₇ is hydrogen.

In another embodiment,

R₁ is a C₁-C₃ alkyl, preferably methyl, or is absent;

R₂ is selected from the group consisting of hydrogen, a hydroxyl group, a 4-dimethylamino-phenyl group, a 4-methylamino-phenyl group and an aminophenyl group, preferably from hydrogen, a hydroxyl group and a 4-dimethylamino-phenyl group;

R₃ is a hydroxyl group or —C(═O)R₈ with R₈ being a C₁-C₃ alkyl, preferably methyl, or —CH₂OH;

R₄ is hydrogen, an acetoxy group or a propynyl group, preferably a 1-propynyl group;

R₅ and R₆ are hydrogen; and

R₇ is hydrogen or methyl.

In another embodiment,

R₁ is absent;

R₂ is selected from the group consisting of a 4-dimethylamino-phenyl group, a 4-methylamino-phenyl group and an aminophenyl group, preferably a 4-dimethylamino-phenyl group;

R₃ is a hydroxyl group;

R₄ is a propynyl group, preferably a 1-propynyl group;

R₅ and R₆ are hydrogen; and

R₇ is hydrogen.

In a further embodiment, the compound of formula (I) is mifepristone or a metabolite or analogue thereof.

Preferably, in this embodiment,

R₁ is absent;

R₂ is a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;

R₃ is a hydroxyl group or —O—C(═O)R₁₁ with R₁₁ being a C₁-C₆ alkyl group optionally substituted by a carboxyl group;

R₄ is a C₁-C₆ alkyl, C₂-C₆ alkenyl or C₂-C₆ alkynyl group optionally substituted by a hydroxyl group or a halogen; or

R₃ and R₄ taken together form tetrahydrofuran group optionally substituted by a methylene group;

R₅ and R₆ are hydrogen;

R₇ is hydrogen.

Preferably, R₂ is a phenyl group substituted, preferably in para position, by a dimethylamino group, a methylamino group, an amino group, a thiomethyl group, a dimethylamino N-oxide group, or —C(═O)R₁₀ with R₁₀ being a methyl. More preferably, R₂ is a phenyl group substituted, preferably in para position, by a dimethylamino group, a methylamino group or an amino group.

Preferably, R₃ is a hydroxyl group or —O—C(═O)R₁₁ with R₁₁ being an ethyl group optionally substituted by a carboxyl group. More preferably, R₃ is a hydroxyl group.

Preferably, R₄ is a C₂-C₃ alkyl group, C₂-C₃ alkenyl or C₂-C₃ alkynyl group optionally substituted by a hydroxyl group or a halogen, preferably chlorine. More preferably, R₄ is a C₃ alkyl, C₃ alkenyl or C₃ alkynyl group, preferably 1-propynyl, 1-propenyl or propyl group, optionally substituted by a hydroxyl group or a halogen.

R₄ may be a 1-propynyl group, 1-propenyl group or a propyl group optionally substituted by a hydroxyl group, preferably a 1-propynyl group optionally substituted by a hydroxyl group.

Alternatively, R₃ and R₄ taken together may form tetrahydrofuran group substituted by a methylene group.

In a particular embodiment,

R₁ is absent;

R₂ is a phenyl group substituted, preferably in para position, by a dimethylamino group, a methylamino group, an amino group;

R₃ is a hydroxyl group;

R₄ is a C₁-C₆ alkyl, C₂-C₆ alkenyl or C₂-C₆ alkynyl group optionally substituted by a hydroxyl group or a halogen, preferably a C₃ alkyl, C₃ alkenyl or C₃ alkynyl group, in particular 1-propynyl, 1-propenyl or propyl group, optionally substituted by a hydroxyl group or a halogen;

R₅ and R₆ are hydrogen;

R₇ is hydrogen.

In a particular embodiment, the compound of formula (I) is selected from the group consisting of

| Name | Formula |
|---|---|
| Corticosterone | 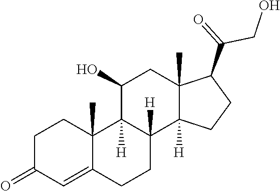 |
| Progesterone | 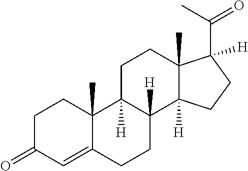 |
| Melengestrol acetate | 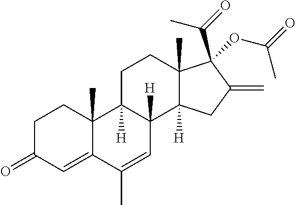 |
| Megestrol acetate | 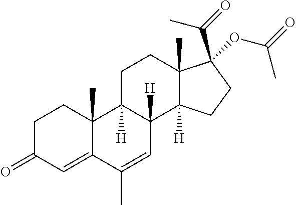 |
| Nestorone | 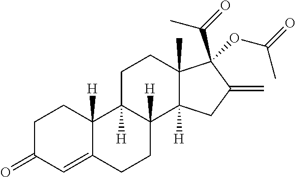 |
| Mifepristone | 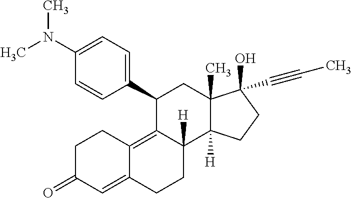 |
| RU42633 | 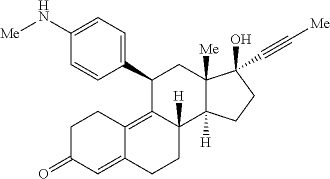 |
| RU42848 | 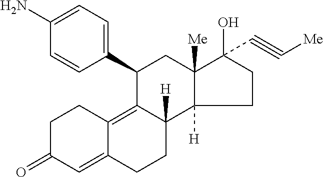 |
| RU42698 | 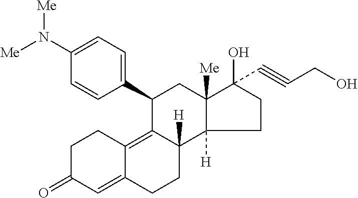 |
| Lilopristone | 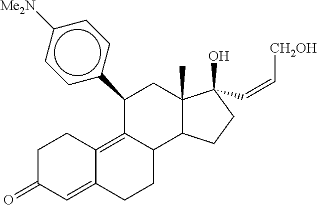 |
| Onapristone | 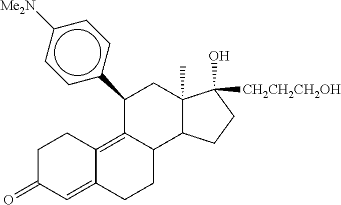 |
| ORG 31710 | 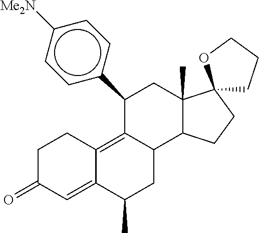 |
| ORG 33628 | 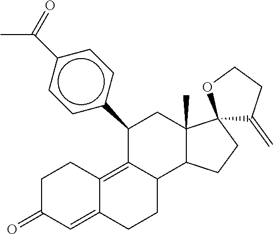 |
| RU 46556 | 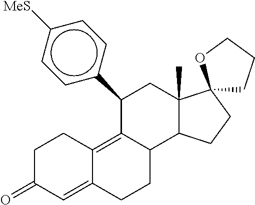 |
| RU 39973 | 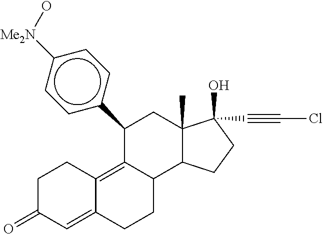 |
| RU 52562 | 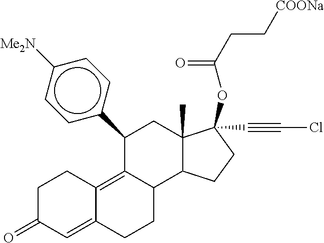 |
| Aglepristone | 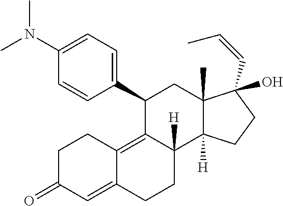 |

or any pharmaceutically acceptable diastereoisomer, salt, hydrate, solvate or prodrug thereof.

In a particular embodiment, the compound of formula (I) may be selected from the group consisting of corticosterone, progesterone, melengestrol acetate, megestrol acetate, nestorone and mifepristone, or any pharmaceutically acceptable salt, hydrate, solvate or prodrug thereof.

The compound of formula (I) may also be selected from the group consisting of corticosterone, megestrol acetate, melengestrol acetate and nestorone, preferably from corticosterone, melengestrol acetate and nestorone, or any pharmaceutically acceptable salt, hydrate, solvate or prodrug thereof.

The compound of formula (I) may also be selected from the group consisting of corticosterone, melengestrol acetate, nestorone and mifepristone, preferably from corticosterone, nestorone and mifepristone, more preferably from nestorone and mifepristone, or any pharmaceutically acceptable salt, hydrate, solvate or prodrug thereof.

In a preferred embodiment, the compound of formula (I) is selected from the group consisting of corticosterone, nestorone, mifepristone and its metabolites and analogues, preferably from nestorone, mifepristone and its metabolites and analogues, or any pharmaceutically acceptable salt, hydrate, solvate or prodrug thereof.

In a particularly preferred embodiment, the compound of formula (I) is selected from the group consisting of mifepristone and its metabolites and analogues.

As used herein, the term “analogues of mifepristone” refers to compounds having formula (I) wherein

R₁ is absent;

R₂ is a phenyl group optionally substituted, preferably in para position;

R₅ and R₆ are hydrogen; and

R₇ is hydrogen,

and having substantially the same biological activity, i.e. a high affinity for the progesterone receptor.

As used herein, the term “metabolites of mifepristone” refers to compounds having formula (I) wherein

R₁ is absent;

R₂ is a phenyl group optionally substituted, preferably in para position;

R₅ and R₆ are hydrogen; and

R₇ is hydrogen,

and having substantially the same biological activity, i.e. a high affinity for the progesterone receptor and which can be obtained through enzyme-catalyzed reactions that occur naturally within cells.

Preferably, metabolites of mifepristone are selected from RU42633, RU42848 and RU42698, or any pharmaceutically acceptable salt, hydrate, solvate or prodrug thereof.

Preferably, analogues of mifepristone are selected from lilopristone, onapristone, aglepristone, ORG 31710, ORG 33628, RU 46556, RU 39973 and RU 52562 (Hazra and Pore, J. Indian Inst. Sci. 2001, 81, 287-298), or any pharmaceutically acceptable salt, hydrate, solvate or prodrug thereof.

In a particular embodiment, the compound of formula (I) is selected from the group consisting of mifepristone, RU42633, RU42848 and RU42968, preferably from the group consisting of mifepristone, RU42633 and RU42848, and any pharmaceutically acceptable salt, hydrate, solvate or prodrug thereof. In a preferred embodiment, the compound of formula (I) is mifepristone, or a pharmaceutically acceptable salt, hydrate, solvate or prodrug thereof.

The compounds of formula (I) as described above may be used in the form of pharmaceutically acceptable diastereoisomers, salts, hydrates and solvates, preferably in the form of pharmaceutically acceptable salts, hydrates and solvates.

Said pharmaceutically acceptable salts, hydrates and solvates of Formula (I) compounds may be formed, where appropriate, by methods well known to those of skill in the art.

The term “pharmaceutically acceptable salt” refers to salts which are non-toxic for a patient and suitable for maintaining the stability of a therapeutic agent and allowing the delivery of said agent to target cells or tissue. Pharmaceutically acceptable salts are well known in the art.

As used herein, the term “solvate” refers to a solvent addition form that contains either stoichiometric or non stoichiometric amounts of solvent. Some compounds have a tendency to trap a fixed molar ratio of solvent molecules in the crystalline solid state, thus forming a solvate. If the solvent is water, the solvate formed is a hydrate. When the solvent is alcohol, the solvate formed is an alcoholate. Hydrates are formed by the combination of one or more molecules of water with one of the substances in which the water retains its molecular state as H₂O, such combination being able to form one or more hydrates.

The compounds of formula (I) as described above may also be used in the form of a prodrug. Prodrugs are generally drug precursors that, following administration to an individual and subsequent absorption, are converted to an active, or a more active species via some process, such as conversion by a metabolic pathway. Some prodrugs have a chemical group present on the prodrug that, for example, renders it less active, increases its solubility and/or improves safety profiles over administration of the parent drugs. In some instances, the prodrugs may be less susceptible to in vivo degradation and exhibit a greater half-life than its parent drug. Once the chemical group has been cleaved and/or modified from the prodrug, the active drug is generated. Prodrugs are often useful because, in some situations, they are easier to administer than the parent drug. They are, for instance, bioavailable by oral administration whereas the parent is not. In certain instances, the prodrug also has improved solubility in pharmaceutical compositions over the parent drug. An example, without limitation, of a prodrug would be a compound as described herein which is administered and subsequently subjected to a biotransformation in vivo and thus provides a therapeutically effective concentration of an active agent. For further general examples, see: Bundgaard, “Design and Application of Prodrugs” in A Textbook of Drug Design and Development, Krosgaard-Larsen and Bundgaard, Ed., 1991, Chapter 5, 113-191, which is incorporated herein by reference. Prodrugs may be prepared, for example, by modifying functional groups present in the compound in such a way that the modifications are cleaved, either in routine manipulation or in vivo, to the parent compound.

The present invention also relates to a pharmaceutical composition comprising a compound of formula (I) according to the invention and as described above, or any pharmaceutically acceptable salt, hydrate, solvate or prodrug thereof, and a pharmaceutically acceptable carrier and/or excipient, preferably for use as skeletal muscle hypertrophy inducer.

All embodiments described above for the compounds of formula (I) as skeletal muscle hypertrophy inducers are also encompassed in this aspect.

The pharmaceutical composition of the invention is formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

Possible pharmaceutical compositions include those suitable for oral, transmucosal (including nasal, rectal or vaginal), topical (including transdermal, buccal and sublingual), or parenteral (including subcutaneous, intramuscular, intravenous and intradermal) administration. For these formulations, conventional excipient can be used according to techniques well known by those skilled in the art. Preferably, the pharmaceutical composition of the invention is suitable for oral administration.

The compositions for parenteral administration are generally physiologically compatible sterile solutions or suspensions which can optionally be prepared immediately before use from solid or lyophilized form. Adjuvants such as a local anesthetic, preservative and buffering agents can be dissolved in the vehicle and a surfactant or wetting agent can be included in the composition to facilitate uniform distribution of the active ingredient.

For oral administration, the composition can be formulated into conventional oral dosage forms such as tablets, capsules, powders, granules and liquid preparations such as syrups, elixirs, and concentrated drops. Non toxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical composition according to the invention may be formulated to release the active drug substantially immediately upon administration or at any predetermined time or time period after administration.

Pharmaceutical composition according to the invention can comprise one or more compound of formula (I) of the invention and as described above associated with one or several pharmaceutically acceptable excipients and/or carriers. These excipients and/or carriers are chosen according to the form of administration as described above.

Pharmaceutical composition according to the invention may also comprise one or several additional active compounds. Said additional active compounds may be selected, for example, from the group consisting of anti-inflammatories, protein anabolic agents (e.g. growth hormone or insulin-like growth factor I), antineoplastic agents, antibiotics, local anesthetics, anabolic/androgenic steroids (e.g. testosterone), glucocorticoids, appetite stimulants (e.g. dronabinol), cytokine modulators (e.g. thalidomide), angiotensin and beta-adrenoreceptor inhibitors, NHE-1 inhibitors (e.g. rimeporide), antifibrotic drugs (e.g. losartan or Lisinopril), phosphodiesterase 5 (PDE5) inhibitors (e.g tadalafil or sildenafil), dehydroepiandrosterone, Vitamin D, ursolic acid, omega 3 acids, angiotensin-converting enzyme (ACE) inhibitors, proteasome inhibitors, cyclophilin D inhibitors, PGC-1 a (alpha) pathway modulators, myostatin and activin A antagonists, ghrelin agonists, β2-adrenoreceptor agonists, creatine supplements, antifibrotic drugs such as losartan and lisinopril, muscle ischemia therapies such as tadalafil and sildenafil, mutation specific therapies such as exon skipping therapies (e.g. eteplirsen, a morpholino phosphorodiamidate antisense oligomer targeting mutations implicated in DMD cases), and agents for therapeutic nonsense suppression such as ataluren, utrophin upregulators such as SMT-C1100.

In the experimental section, the inventors demonstrated that compounds of formula (I) according to the invention and as described above, are able to promote the differentiation of myoblasts into myotubes, to increase the number and size of myotubes, and/or to increase the fusion index reflecting the capacity of cells to regenerate.

Accordingly, the present invention relates to a compound of formula (I) or any pharmaceutically acceptable salt, hydrate, solvate or prodrug thereof, or a pharmaceutical composition according to the invention and as described above, for use as skeletal muscle hypertrophy inducer.

Skeletal muscle fibers are syncytia that arise from the sequential fusion of myoblast cells. The process involves i) the differentiation of myoblasts into myocytes, ii) the fusion of myocytes to form nascent myotubes and iii) additional fusion of myocytes with nascent myotubes to form more mature myotubes. Accordingly, as used herein, the expression «skeletal muscle hypertrophy» refers to a gain of skeletal muscle mass characterized by an increase in the size of pre-existing myofibers and/or an increase in the number of myofibers and/or an increase in the mean number of nuclei per myotube and/or an increase in the fusion index (number of nuclei in myotubes divided by total number of nuclei in myoblasts and myotubes). Preferably, the expression «skeletal muscle hypertrophy» refers by an increase in the size of pre-existing myofibers and/or an increase in the number of myofibers and/or an increase in the fusion index. As used herein, the terms “myotube” and “myofiber” are used interchangeably.

The present invention also relates to a method for inducing skeletal muscle hypertrophy in a subject in need thereof, comprising administering a therapeutically effective amount of a compound of formula (I) or any pharmaceutically acceptable salt, hydrate, solvate or prodrug thereof, or a pharmaceutical composition according to the invention and as described above, to said subject.

The therapeutically effective amount to be administered may be easily chosen by the skilled person and should be sufficient to provide an increase of skeletal muscle mass or skeletal muscle strength in the subject.

As used herein, the subject is an animal, preferably a mammal, more preferably a human being. Preferably, the subject is a subject suffering from muscle wasting or weakness resulting from a disease or disorder resulting in loss of skeletal muscle tissue and/or skeletal muscle weakness, such as diseases or disorders described below.

The present invention further concerns the use of a compound of formula (I), or any pharmaceutically acceptable salt, hydrate, solvate or prodrug thereof, or a pharmaceutical composition according to the invention and as described above, for preparing a medicament inducing skeletal muscle hypertrophy.

The present invention also relates to a compound of formula (I) or any pharmaceutically acceptable salt, hydrate, solvate or prodrug thereof, or a pharmaceutical composition according to the invention and as described above, for use to prevent involuntary loss of skeletal muscle mass, preferably due to the degeneration of muscle fibers, for use to promote or stimulate skeletal muscle mass increase, for use to replete skeletal muscle mass and/or for use to increase skeletal muscle mass and/or strength.

The present invention also relates to a method for preventing involuntary loss of skeletal muscle mass, preferably due to the degeneration of muscle fibers, promoting or stimulating skeletal muscle mass increase, repleting skeletal muscle mass and/or increasing skeletal muscle mass and/or strength, in a subject in need thereof, comprising administering a therapeutically effective amount of a compound of formula (I) or any pharmaceutically acceptable salt, hydrate, solvate or prodrug thereof, or a pharmaceutical composition according to the invention and as described above, to said subject.

The therapeutically effective amount to be administered may be easily chosen by the skilled person and should be sufficient to prevent involuntary loss of skeletal muscle mass, to promote or stimulate skeletal muscle mass increase, to replete skeletal muscle mass and/or to increase skeletal muscle mass and/or strength.

The subject may be as defined above.

The present invention further concerns the use of a compound of formula (I), or any pharmaceutically acceptable salt, hydrate, solvate or prodrug thereof, or a pharmaceutical composition according to the invention and as described above, for preparing a medicament preventing involuntary loss of skeletal muscle mass, preferably due to the degeneration of muscle fibers, promoting or stimulating skeletal muscle mass increase, repleting skeletal muscle mass and/or increasing skeletal muscle mass and/or strength.

In the experimental section, the inventors demonstrated that compound of formula (I) according to the invention and described above, are not only able to promote the differentiation of myoblasts into myotubes and to increase the fusion index reflecting the capacity of cells to regenerate, but are also able to prevent skeletal muscle atrophy, in particular atrophy induced by IL-1β, TNF-α or myostatin.

Thus, the present invention also relates to a compound of formula (I) or any pharmaceutically acceptable salt, hydrate, solvate or prodrug thereof, or a pharmaceutical composition according to the invention and as described above, for use to promote skeletal muscle regeneration and/or prevent skeletal muscle atrophy.

All embodiments described above for the compounds of formula (I) as skeletal muscle hypertrophy inducers are also encompassed in this aspect.

As used herein, the expression “skeletal muscle regeneration” refers to the capacity of muscle cells or tissue to regenerate, i.e. to produce new myotubes from myoblasts. The expression “to promote skeletal muscle regeneration” thus refers to the capacity of compounds of formula (I) to promote differentiation of myoblasts into myotubes and/or to increase the number of myotubes and/or to improve the regeneration capacity of muscle tissue and in particular of myotubes.

As used herein, the expression “to prevent skeletal muscle atrophy” refers to the capacity of compounds of formula (I) to prevent, stop or slow down muscle wasting. Muscle atrophy may be caused for example by a disease state, a particular physiological condition such as aging, food starvation or inactivity, or an atrophying agent such as drug (statins) or poison (botulinum toxin). Prevention of muscle atrophy is preferably obtained by increasing the production of muscle mass and then counter balancing muscle loss.

The present invention also relates to a method for promoting skeletal muscle regeneration and/or preventing skeletal muscle atrophy in a subject in need thereof, comprising administering a therapeutically effective amount of a compound of formula (I) or any pharmaceutically acceptable salt, hydrate, solvate or prodrug thereof, or a pharmaceutical composition according to the invention and as described above, to said subject.

The therapeutically effective amount to be administered may be easily chosen by the skilled person and should be sufficient to stimulate skeletal muscle regeneration and/or prevent, stop or slow down muscle wasting, preferably by increasing the production of muscle mass and then counter balancing muscle loss.

The subject may be as defined above.

The present invention further relates to the use of a compound of formula (I) or any pharmaceutically acceptable salt, hydrate, solvate or prodrug thereof, or a pharmaceutical composition according to the invention and as described above, for preparing a medicament for promoting skeletal muscle regeneration and/or preventing skeletal muscle atrophy.

The present invention further relates to a compound of formula (I) or any pharmaceutically acceptable salt, hydrate, solvate or prodrug thereof, or a pharmaceutical composition according to the invention and as described above, for use in the treatment or prevention of muscle wasting, and in particular in the treatment or prevention of a disease or disorder resulting in loss of skeletal muscle tissue and/or skeletal muscle weakness.

It also concerns the use of a compound of formula (I) or any pharmaceutically acceptable salt, hydrate, solvate or prodrug thereof, or a pharmaceutical composition according to the invention and as described above, for preparing a medicament for treating muscle wasting, and in particular a disease or disorder resulting in loss of skeletal muscle tissue and/or skeletal muscle weakness.

It finally concerns a method for treating muscle wasting, and in particular a disease or disorder resulting in loss of skeletal muscle tissue and/or skeletal muscle weakness, in a subject in need thereof, comprising administering a therapeutically active amount of a compound of formula (I) or any pharmaceutically acceptable salt, hydrate, solvate or prodrug thereof, or a pharmaceutical composition according to the invention and as described above, to the subject.

All embodiments described above for the compounds of formula (I) as skeletal muscle hypertrophy inducers are also encompassed in this aspect.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

As used herein, the term “treatment of muscle wasting” may refer to the therapy, prevention or retardation of involuntary loss of skeletal muscle mass, preferably due to the degeneration of muscle fibers.

In particular, the term “treatment of a disease or disorder resulting in loss of skeletal muscle tissue and/or skeletal muscle weakness” may refer to a preservation or increase of the skeletal muscle mass and/or the skeletal muscle strength of a patient or a slow-down of the skeletal muscle mass loss and/or the skeletal muscle strength loss of a patient.

The effective amount may be a therapeutically or prophylactically effective amount. A “therapeutically effective amount” refers to an amount effective, at dosages and for periods of time necessary, to achieve the desired therapeutic or prophylactic result. The therapeutically effective amount may vary according to factors such as the disease or disorder, disease state, age, sex, and weight of the individual. A therapeutically effective amount encompasses an amount in which any toxic or detrimental effects are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The disease or disorder to be treated may be any disease or disorder resulting in loss of skeletal muscle tissue or mass and/or skeletal muscle weakness.

Muscle wasting (i.e. loss of skeletal muscle tissue) and weakness may result from a large panel of diseases or disorders such as metabolic diseases (e.g. glycogen storage diseases, lipid storage diseases or disorders of purine nucleotide metabolism), neurologic diseases (e.g. Hereditary Sensory and Motor Neuropathies type III) and neuromuscular diseases, cachexia (i.e. muscle atrophy resulting from diseases such as cancer, AIDS, congestive heart failure, chronic obstructive pulmonary disease, severe burns, renal failure or liver failure), sarcopenia, muscle disuse atrophy (i.e. atrophy caused by prolonged inactivity), atrophy induced by excessive food starvation such as starvation due to anorexia nervosa, or muscle injuries including acute muscular injury, muscle overuse injury or wound war injuries.

Preferably, the disease or disorder to be treated is selected from neuromuscular diseases, cachexia, sarcopenia, muscle disuse atrophy, atrophy induced by anorexia food starvation, and muscle injuries including acute muscular injury or muscle overuse injury. More preferably, the disease or disorder to be treated is selected from neuromuscular diseases, cachexia and sarcopenia.

In a particular embodiment, the disease or disorder is a neuromuscular disease, preferably selected from muscle diseases (i.e. myopathies), neuromuscular junction diseases or motor neuron diseases.

Myopathies are neuromuscular disorders in which the primary symptom is muscle weakness due to dysfunction of skeletal muscle fibres. Myopathies can be inherited or acquired and include, for example, muscular dystrophies, metabolic myopathies such as mitochondrial myopathies or drug-induced myopathies, and autoimmune myopathies such as dermatomyositis, polymyositis or inclusion body myositis.

Muscular dystrophies represent a large group of myopathies causing a progressive degeneration of myofibers and resulting in a loss of muscle mass. Mutations in over 30 genes causing muscular dystrophies have been identified. Examples of muscular dystrophies include, but are not limited to Duchenne muscular dystrophy, Becker muscular dystrophy, congenital muscular dystrophies, facioscapulohumeral muscular dystrophies, myotonic muscular dystrophies, distal muscular dystrophies such as Miyoshi muscular dystrophy, Emery-Dreifuss muscular dystrophy, limb-girdle muscular dystrophies and oculopharyngeal muscular dystrophies.

Motor neuron diseases are disorders which are characterized by the gradual degeneration and death of motor neurons which control voluntary muscles. Motor neurons thus stop sending messages to muscles which gradually weaken and atrophy. Motor neuron diseases include, for example, amyotrophic lateral sclerosis, primary lateral sclerosis, progressive muscular atrophy, progressive bulbar palsy, pseudobulbar palsy and spinal muscular atrophies.

Neuromuscular junction diseases are disorders which have in common the perturbation of the neurotransmission through the neuromuscular junction and result in progressive weakness due to a reduced muscle strength. Neuromuscular junction diseases include, for example, myasthenia gravis, autoimmune neuromyotonia (Isaacs' syndrome), Lambert-Eaton myasthenic syndrome, or may result of a form of poison that effects neuromuscular junction functioning such as snake venom or neurotoxins (e.g Clostridium botulinum toxin).

Preferably, the neuromuscular disease is selected from muscular dystrophies, and in particular from Duchenne muscular dystrophy, Becker muscular dystrophy, myotonic muscular dystrophies, distal muscular dystrophies such as Miyoshi muscular dystrophy, and limb-girdle muscular dystrophies.

In another particular embodiment, the disease or disorder is selected from cachexia and sarcopenia, preferably is sarcopenia.

In the methods of the present invention, the compound of formula (I) or any pharmaceutically acceptable salt, hydrate, solvate or prodrug thereof, or a pharmaceutical composition according to the invention and as described above, may be used in combination with other active ingredients that can be chosen according to the disease to be prevented or treated. Examples of other active ingredients include, but are not limited to, anti-inflammatories, protein anabolic agents (e.g. growth hormone or insulin-like growth factor I), antineoplastic agents, antibiotics, local anesthetics, anabolic/androgenic steroids (e.g. testosterone), glucocorticoids, appetite stimulants (e.g. dronabinol), cytokine modulators (e.g. thalidomide), angiotensin and beta-adrenoreceptor inhibitors, NHE-1 inhibitors (e.g. rimeporide), antifibrotic drugs (e.g. losartan or Lisinopril), phosphodiesterase 5 (PDE5) inhibitors (e.g tadalafil or sildenafil), dehydroepiandrosterone, Vitamin D, ursolic acid, omega 3 acids, angiotensin-converting enzyme (ACE) inhibitors, proteasome inhibitors, cyclophilin D inhibitors, PGC-1 a (alpha) pathway modulators, myostatin and activin A antagonists, ghrelin agonists, β2-adrenoreceptor agonists, creatine supplements, antifibrotic drugs such as losartan and lisinopril, muscle ischemia therapies such as tadalafil and sildenafil, mutation specific therapies such as exon skipping therapies (e.g. eteplirsen, a morpholino phosphorodiamidate antisense oligomer targeting mutations implicated in DMD cases), agents for therapeutic nonsense suppression such as ataluren, utrophin upregulators such as SMT-C1100, gene replacement therapies (such as using rAAV2.5-CMV-Mini-dystrophy, rAAVrh74.MCK.Mini-dystrophy or rAAV1.CMV.huFollistatin344) or cell therapies using muscle precursor cells or stem cells.

Such combination therapies noted above encompass combined administration (where two or more therapeutic agents are included in the same or separate formulations), and separate administration, in which case, administration of the compound of formula (I) can occur prior to, simultaneously, and/or following, administration of the additional therapeutic agent.

The compound of formula (I) (and any additional therapeutic agent) can be administered by any suitable means, including parenteral, oral, transmucosal or topical administration, preferably oral administration.

The doses used for the administration can be adapted as a function of various parameters, and in particular as a function of the mode of administration used, of the relevant pathology, or alternatively of the desired duration of treatment. The compound of formula (I) (and any additional therapeutic agent) may be administered as a single dose or in multiple doses.

The amount of compound of formula (I) which will be effective in the treatment of a particular disorder or condition will depend on the nature of the disorder or condition, and can be determined by standard clinical techniques.

In a preferred embodiment, each dose may range from about 0.05 mg to about 100 mg per kilogram of body weight of compound of formula (I), preferably from about 0.1 mg to about 50 mg per kilogram of body weight, and more preferably from about 0.25 mg to about 10 mg per kilogram of body weight of compound of formula (I).

The dosing schedule for administration may vary from once a month to daily depending on a number of clinical factors, including the type of disease, severity of disease, and the subject's sensitivity to the therapeutic agent.

As shown in the experimental section, the compounds of formula (I) of the invention and as described above are able to prevent muscle atrophy.

The present invention thus also concerns a product containing a compound of formula (I) or any pharmaceutically acceptable salt, hydrate, solvate or prodrug thereof, as described above and a compound inducing skeletal muscular atrophy, as a combined preparation for simultaneous, separate or sequential use.

All embodiments described above for the compounds of formula (I) as skeletal muscle hypertrophy inducers are also encompassed in this aspect.

In a particular embodiment, the compound inducing skeletal muscular atrophy is a therapeutic agent. In this embodiment, the compound of formula (I) or any pharmaceutically acceptable salt, hydrate, solvate or prodrug thereof, is used to prevent or limit drug-induced myopathy.

The present invention further concerns a method for preventing or limiting the skeletal muscular atrophy induced by a therapeutic agent in a subject comprising administering a therapeutically effective amount of a compound of formula (I) or any pharmaceutically acceptable salt, hydrate, solvate or prodrug thereof, as described above to said subject simultaneously, separately or sequentially to the administration of said therapeutic agent inducing skeletal muscular atrophy.

Examples of therapeutic agents inducing skeletal muscular atrophy include, but are not limited to corticosteroids, colchicine, chloroquine, hydroxychloroquine, D-penicillamine, antibiotics, betablockers, amiodarone, cimetidine, zidovudine, vincristine, clofibrate, statins, fibrates, cyclosporine, L-tryptophan, drugs causing hypokalaemia and lipid lowering agents, or combinations of drugs such as a fibrate and a statin or cyclosporin and colchicin, and therapeutic agents administered by intramuscular route such as vaccines.

In a preferred embodiment, the therapeutic agent inducing skeletal muscular atrophy include is a lipid lowering agent, preferably selected from statins and fibrates.

The compound of formula (I) and the therapeutic agent inducing skeletal muscular atrophy may be administered simultaneously. Alternatively, the compound of formula (I) may be administered to the subject prior or after administration of the therapeutic agent inducing skeletal muscular atrophy. Preferably, when the therapeutic agent and the compound of formula (I) are administered separately, they are both administered within 24 hours.

Compounds of formula (I) of the invention and as described above, i.e. skeletal muscle hypertrophy inducers, may also find applications in feed and food industries, in particular as dietary supplements.

Accordingly, in a further aspect, the present invention also relates to a dietary supplement composition comprising a compound of formula (I) as defined above, i.e. a compound of formula (I)

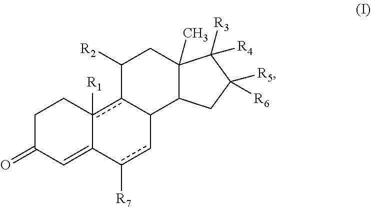

preferably of formula (I)

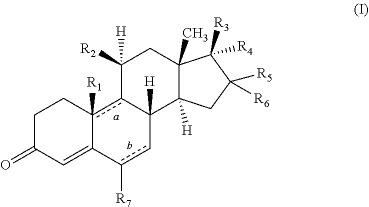

wherein

R₁ is hydrogen or a C₁-C₃ alkyl, or is absent;

R₂ is selected from the group consisting of hydrogen, a hydroxyl group, a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;

R₃ is a hydroxyl group, —C(═O)R₈ with R₈ being a C₁-C₃ alkyl optionally substituted by a hydroxyl group, or —O—C(═O)R₁₁ with R₁₁ being a C₁-C₆ alkyl group optionally substituted by a carboxyl group;

R₄ is hydrogen, an acetoxy group or a C₁-C₆ alkyl, C₂-C₆ alkenyl or C₂-C₆ alkynyl group optionally substituted by a hydroxyl group or a halogen; or

R₃ and R₄ taken together form a tetrahydrofuran group optionally substituted by a methylene group;

R₅ and R₆ are hydrogen or taken together form a methylene group;

R₇ is hydrogen or methyl

a and b respectively denote, independently from each other, a single bond or a double bond, with the proviso that R₁ is absent when a is a double bond;

with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group,

or any pharmaceutically acceptable diastereoisomer, salt, hydrate, solvate or prodrug thereof.

In particular embodiment, the compound is a compound of formula (I)

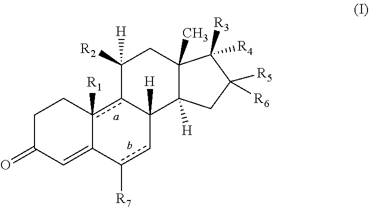

wherein

R₁ is hydrogen or a C₁-C₃ alkyl, or is absent;

R₂ is selected from the group consisting of hydrogen, a hydroxyl group, a 4-dimethylamino-phenyl group, a 4-methylamino-phenyl group and an aminophenyl group;

R₃ is a hydroxyl group or —C(═O)R₈ with R₈ being a C₁-C₃ alkyl or —CH₂OH;

R₄ is hydrogen, an acetoxy group or a propynyl group;

R₅ and R₆ are hydrogen or taken together form a methylene group;

R₇ is hydrogen or methyl;

a and b respectively denote, independently from each other, a single bond or a double bond, with the proviso that R₁ is absent when a is a double bond; and

with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group,

or any acceptable salt, hydrate, solvate or prodrug thereof.

It also relates to a non-therapeutic use of a compound of formula (I) as defined above, or of a dietary supplement composition of the invention to increase muscle mass, muscle strength and/or muscle performance in a subject. It further relates to a non-therapeutic use of a compound of formula (I) as defined above, or of a dietary supplement composition of the invention for use to prevent loss of skeletal muscle mass, preferably involuntary and/or undesired loss of skeletal muscle mass.

The subject is preferably a mammal, more preferably a human being.

In an embodiment, the subject is a non-human animal, preferably a mammal, and even more preferably a livestock animal or a sports or leisure animal, e.g. racehorses. Livestock animals are non-human mammals, preferably mammals used for meat. In particular livestock animals may be selected from pig, cattle, goat, sheep, horse, bison, deer, elk or moose.

In another embodiment, the subject is a human being, preferably an adult human.

In a particular embodiment, the subject is an older adult human, e.g. of more than 60, and the dietary supplement composition is used, or is suitable, to stop, slow/down or prevent muscle function and/or mass decline.

The subject is preferably a healthy subject, i.e. a subject who is not suffering from a disease or disorder resulting in loss of skeletal muscle tissue and/or muscle weakness.

The dietary supplement composition may be in the form of a powder, liquid, or solid.

Preferably, the dietary supplement composition is formulated for oral administration. In particular, said dietary supplement composition may be formulated into conventional oral dosage forms such as tablets, capsules, powders, granules and liquid preparations such as syrups, elixirs, and concentrated drops. Non toxic solid carriers or diluents may be used which include, for example, mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

The dietary supplement composition may comprise further ingredient providing beneficial effects to the subject such as vitamins (e.g. vitamin D), amino acids, proteins, lipids (omega 3 fatty acids), ursolic acid, tomaditine, antioxidants, polyphenols, isoflavones present in soybean and derivatives, tea leaves components and garlic compounds.

All embodiments described above for the compounds of formula (I) and their uses, in particular, as skeletal muscle hypertrophy inducers are also encompassed in this aspect.

The present invention also relates to the use of a compound of formula (I) as defined above, or any acceptable salt, hydrate, solvate or prodrug thereof, as ingredient for animal feed composition or as additive for animal feed composition. It also relates to the use of a compound of formula (I) as defined above, or any acceptable salt, hydrate, solvate or prodrug thereof, to prepare an ingredient or additive for animal feed composition. It further relates to an ingredient or additive for animal feed composition comprising a compound of formula (I) as defined above, or any acceptable salt, hydrate, solvate or prodrug thereof.

It further relates to a feed composition for livestock comprising a compound of formula (I) as defined above, or any acceptable salt, hydrate, solvate or prodrug thereof, as ingredient or additive.

The feed composition, ingredient, additive, or dietary supplement of the invention may further comprise any edible GRAS (generally recognized as safe) material such as, for example, corn gluten feed, sunflower hulls, distillers grains, guar hulls, wheat middlings, rice hulls, rice bran, oilseed meals, dried blood meal, animal by-product meal, fish by-product, fish meal, dried fish solubles, feather meal, poultry by-products, meat meal, bone meal, dried whey, soy protein concentrate, soy flour, yeast, wheat, oats, grain sorghums, corn feed meal, rye, corn, barley, aspirated grain fractions, brewers dried grains, corn flour, corn gluten meal, feeding oat meal, sorghum grain flour, wheat mill run, wheat red dog, hominy feed, wheat flour, wheat bran, wheat germ meal, oat groats, rye middlings, cotyledon fiber, ground grains, or a mixture thereof.

Preferably, the feed composition, ingredient, additive, or dietary supplement of the invention is used as non-therapeutic skeletal muscle hypertrophy inducer, and in particular to improve livestock performance, i.e. to increase liveweight gain. Thus, preferably, the feed composition, ingredient, additive, or dietary supplement is intended to be administered to a healthy subject, i.e. a subject who is not suffering from a disease or disorder resulting in loss of skeletal muscle tissue and/or muscle weakness. The subject may be as defined above for the dietary supplement composition of the invention.

The invention also relates to a method of improving livestock performance and/or health comprising providing to said livestock a compound of formula (I) as defined above, or any acceptable salt, hydrate, solvate or prodrug thereof, in particular a feed composition, ingredient, additive, or dietary supplement of the invention. Preferably, as used herein, the term “improving livestock performance” refers to increase liveweight gain. This use is intended to be a non-therapeutic use as explained above and preferably, the compound, feed composition, ingredient, additive, or dietary supplement is intended to be administered to healthy livestock, i.e. who is not suffering from a disease or disorder resulting in loss of skeletal muscle tissue and/or muscle weakness.

The feed composition, ingredient, additive, or dietary supplement may be in the form of a powder, liquid, or solid.

Ingredients of the feed composition of the invention other than the compound of formula (I) depend on the nature of the livestock and may be easily chosen by the skilled person.

Preferably, the feed composition of the invention is in a form and/or a composition approved by a governmental institution such as National Food Administration (for example ANSES in France, ACIA in Canada, or FAD in the US).

All embodiments described above for the compound of formula (I) and its uses, in particular, as skeletal muscle hypertrophy inducers are also encompassed in this aspect.

In a last aspect, the present invention relates to all aspects disclosed above wherein the compound of formula (I) is replaced by a compound selected from mifepristone, and metabolites and analogues thereof.

In particular, the present invention also relates to
a compound selected from mifepristone, and metabolites and analogues thereof for use as skeletal muscle hypertrophy inducer, for use to promote skeletal muscle regeneration and/or prevent skeletal muscle atrophy, or for use in the treatment or prevention of a disease or disorder resulting in loss of skeletal muscle tissue and/or muscle weakness;
a product containing a compound selected from mifepristone, and metabolites and analogues thereof, and a compound inducing skeletal muscular atrophy, as a combined preparation for simultaneous, separate or sequential use;
the non-therapeutic use of a compound selected from mifepristone, and metabolites and analogues thereof, to increase muscle mass, muscle strength and/or muscle performance in a subject, and in particular to increase skeletal muscle mass, skeletal muscle strength and/or skeletal muscle performance in a subject.
the use, preferably the non-therapeutic use, of a compound selected from mifepristone, and metabolites and analogues thereof, to prevent loss of skeletal muscle mass in a subject, or as ingredient or additive for animal feed composition, and
a method of improving livestock performance comprising providing to said livestock a compound selected from mifepristone, and metabolites and analogues thereof, preferably a feed composition, ingredient, additive, or dietary supplement comprising a compound of formula (I) as defined above.

All embodiments described in aspects relating to the compound of formula (I) and its uses, in particular, as skeletal muscle hypertrophy inducers are also encompassed in this aspect, i.e. are to be applied to mifepristone, and metabolites and analogues thereof.

As used herein, the terms “analogues of mifepristone” and “metabolites of mifepristone” refer to compounds having formula (I)

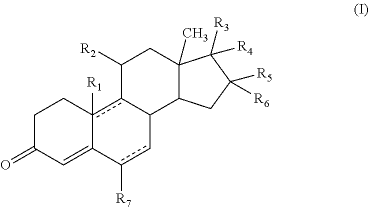

preferably

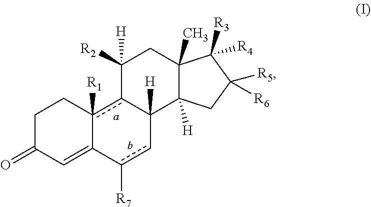

wherein

R₁ is absent;

R₂ is a phenyl group optionally substituted, preferably in para position;

R₅ and R₆ are hydrogen; and

R₇ is hydrogen,

or any pharmaceutically acceptable diastereoisomer, salt, hydrate, solvate or prodrug thereof.

Analogues of mifepristone have substantially the same biological activity than mifepristone, i.e. an affinity for the progesterone receptor and optionally an antiglucocorticoid activity. Preferred analogues have an affinity, preferably a high affinity, for the progesterone receptor and a weak or none antiglucocorticoid activity.

Metabolites of mifepristone have substantially the same biological activity, i.e. an affinity for the progesterone receptor, and can be obtained through enzyme-catalyzed reactions that occur naturally within cells.

Preferably, metabolites of mifepristone are selected from RU42633, RU42848 and RU42698, and any pharmaceutically acceptable diastereoisomer, salt, hydrate, solvate or prodrug thereof.

Preferably, analogues of mifepristone are selected from lilopristone, onapristone, aglepristone, ORG 31710, ORG 33628, RU 46556, RU 39973 and RU 52562 (Hazra and Pore, J. Indian Inst. Sci. 2001, 81, 287-298), and any pharmaceutically acceptable diastereoisomer, salt, hydrate, solvate or prodrug thereof.

In a particular embodiment, the compound of formula (I) is selected from the group consisting of mifepristone, RU42633, RU42848 and RU42968, preferably from the group consisting of mifepristone, RU42633 and RU42848, and any pharmaceutically acceptable diastereoisomer, salt, hydrate, solvate or prodrug thereof. In a preferred embodiment, the compound of formula (I) is mifepristone, or a pharmaceutically acceptable diastereoisomer, salt, hydrate, solvate or prodrug thereof.

The following examples are given for purposes of illustration and not by way of limitation.

### Examples

Materials and Methods

Cell Source and Cell Culture

Healthy donor primary skeletal cells (Donor 1 and Donor 3) were Clonetics™ Human Skeletal Muscle Myoblasts (HSMM).

In addition, cells from a DMD (Duchenne muscular dystrophy) donor were sourced (Donor Z) and used to confirm primary hits.

Donor characteristics are detailed in table 1 below.

TABLE 1
Donor characteristics

| | Donor1 | Donor 3 | Donor Z |
|---|---|---|---|
| Source | Lonza | Lonza | Hospital |
| Donor Age | 16 years | Male | Male |
| Donor Sex | Female | 21 years | 3 years |
| Donor Race | Caucasian | Caucasian | Caucasian |
| Status | Healthy | Healthy | DMD deletion of |
| | | | exon 48 to 52 |
| Desmin positive cells | >90% | >90% | 10% |

Muscle cells were maintained in culture following the supplier instructions with supplements and fetal bovine serum (FBS) serum provided by Lonza. An amplification step was performed in order to obtain enough cells for seeding the screening plates.

Hypertrophy Assay and Atrophy Rescue Assays

Hypertrophy and Atrophy Rescue assays were performed using an in vitro fully automated human myotube model called MyoScreen (Cytoo, France). This model relies on a tight control of the microenvironment that guides the differentiation of human primary myoblasts. Myotubes formed on MyoScreen™ micropatterns present a high level of maturation together with a highly standardized morphology.

Human primary myoblasts from donors 1, 3 and Z were seeded in MyoScreen™ micropatterned 96-well plates (Cytoo, France), let them adhere for 24 h in growth medium, then run the differentiation in a low horse serum medium for at least 5 days.

At Day0, MyoScreen™ plates (Cytoo, France) containing micropatterns were pre-filled with 200 μl/well of growth medium and stored in the incubator at 37° C. Human primary myoblasts were detached from the flasks, count, and seeded into the plates with 15 000 cells per well in 100 μl of growth medium.

At Day 1, the growth medium was changed for a differentiation medium, 300 μl/well (DMEM with 0.1% horse serum) in which myoblasts started differentiating and forming myotubes.

At Day 2, the differentiation medium was changed. Then candidate compounds were diluted with differentiation medium and transferred into the plate. The final concentration of DMSO should be not higher than 0.1%. At least 6 wells were treated with the vehicle as a basal control, and 6 wells were treated with IGF-1 at 100 ng/ml as positive hypertrophy control.

For Atrophy Rescue assay, one hour after candidate compound addition, atrophy inducers were added at the following final concentration: 150 ng/mL of myostatin, 25 ng/mL of IL-13 and 2 ng/mL of TNF-α.

At Day 6, cells were fixed with formalin 5% for 30 min at room temperature, then permeabilized with Triton X-100 at 0.1% in PBS for 15 min, and blocked with PBS+BSA 1% for 20 minutes. Myotubes were incubated with first antibody against Troponin T in blocking buffer for 1 h30, washed three times with PBS, incubated with secondary antibody and Hoescht (1/10 000) for 1 h30, and washed three times with PBS.

Image Analysis

Images were acquired at 10× magnification with an Operetta High Content Imaging System. Image processing and analysis were performed with dedicated algorithms developed on the Acapella High Content Imaging Software (Perkin Elmer) by CYTOO. Eleven fields per well were acquired.

First, segmentation of myotubes and nuclei were done using respectively the Troponin T staining intensity and the Hoechst staining. One to two myotubes per micropattern were usually identified (a myotube is a troponin T staining area that includes at least 2 nuclei). The threshold of segmentation was set-up in order to avoid detecting the background noise and eliminate aberrant small myotube structures. At the end of this first step, specific readouts were calculated in the whole well, like the nuclei count and the fusion index (percentage of nuclei included in troponin T staining). Usually around 50 to 60 myotubes were detected per well in a control condition.

Then, an image clean-up step was performed on the Troponin T images in order to remove myotubes that touch the border of the image. The final valid myotubes were used to extract myotube morphology parameters including the myotube width and area, and the number of nuclei per myotube.

Nuclei Count, Fusion Index, Mean myotube Area and Number of nuclei per myotube have been validated as relevant and sensitive readouts of myotube differentiation as well as atrophic and hypertrophic induction.

Primary Screening

A primary screening was run to identify hypertrophy compounds that increase the myotube differentiation and size. Candidate compounds were tested at 10 μM in monoplicate on Donor 3 cells.

Retest

A retest was run, by cherry picking (same compound batch as primary screening): each hit was tested in the same conditions as in the Primary Screening (Donor 3, 10 μM) in six well replicates.

Dose Response on Two Healthy Donors and a DMD Donor

Dose response assays were performed on three donors (Healthy Donors 3 and 1, DMD Donor Z): 8 doses of candidate compounds between 33 μM and 0.015 μM, 2 well replicates per dose.

EC₅₀ Calculation

Compounds of interest were tested several times in dose response, with triplicate of wells per dose. Results were normalized to the control condition (basal level), and plotted using GraphPadm Prism. The readout “nuclei count” allowed detecting any toxicity effect. The readouts “fusion index” and “myosin area” were used to determine the EC₅₀ value using the GrapgPad Prism fitting solution.

Atrophy Rescue Evaluation

Atrophy Rescue assays were performed in the presence of atrophy inducers, i.e. TNF-α, IL-1β and Myostatin. Each compound was tested in triplicate of wells at 1 μM for nestorone, 3 μM for corticostestrone and 1 μM for mifepristone.

Results

As shown on Table 2 below, six compounds were identified as skeletal muscle hypertrophy inducers during the primary screening, inducing an increase in the fusion index or/and myotube area readouts by more than +30%. These six compounds can be classified into three sub-groups: corticosteroids (corticosterone), hormones including both estrogens and progestogens (Nestorone, Melengestrol, Megestrol, and Progesterone), and one steroid receptor agonist (Mifepristone).

The retest allowed ranking more precisely the activity of each compound compared to the other ones. The most potent compounds from each sub-group were selected for further characterization (Corticosterone, Nestorone and Mifepristone).

TABLE 2
Hypertrophy activity of selected compounds on the healthy donor 3

| | Primary screening | Retest |
|---|---|---|
| Compounds | (% Activity) | (% Activity) |
| Corticosterone | 148 | 128 |
| Nestorone | 153 | 189 |
| Melengestrol acetate | 140 | 140 |
| Megestrol acetate | 133 | 125 |
| Progesterone | 137 | 114 |
| Mifepristone | 140 | 173 |
| IGF-1 (positive control) | 210 | 190 |

(% Activity=Myotube fusion index (compound)*100/Myotube Fusion index (basal control)

Dose responses of the three most potent hits were performed on three donors, two healthy and one DMD. Results are presented in Table 3. The three compounds were active on both healthy male and female donors. Their EC₅₀ could not be defined precisely because the dose response range was too high to detect both minimum and maximum activity levels. Interestingly, Corticosterone and Nestorone compounds induced a significant increase in the DMD myotube size and differentiation.

TABLE 3
Results of the first dose response assay on three different donors

| | Donor 3 | | Donor 1 | | Donor Z | |
|---|---|---|---|---|---|---|
| Compounds | % Activity | EC₅₀ | % Activity | EC₅₀ | % Activity | EC₅₀ |
| Corticosterone | 114 | ~100 nM | 134 | ~1 μM | 145 | ~500 nM |
| Nestorone | 115 | ~100 nM | 130 | ~100 nM | 181 | ~100 nM |
| Mifepristone | 133 | ~50 nM | 145 | ~5 nM | | |
| IGF-1 | 150 | | 150 | | 250 | |

An additional dose response assay was performed with Mifepristone on cells from Donor Z showing an activity of 156% at 300 nM (positive control IGF-1: 200%).

In order to determine the compounds EC₅₀ more precisely, a second dose response was run on the healthy donor 3, testing lower concentrations: results are summarized in Table 4. EC₅₀ values below the micromolar range were confirmed.

TABLE 4
Results of the second dose response assay on the healthy donor 3

Donor 3

| Compounds | % Activity | EC₅₀ |
|---|---|---|
| Corticosterone | 120 | 200 nM |
| Nestorone | 133 | 200 nM |
| Mifepristone | 135 | 100 nM |
| IGF-1 | 150 | |

Atrophy rescue assays were performed for the three most potent hits and results are presented in Table 5. Myotubes from the healthy donor 3 were atrophied using different inducers: inflammation cytokines IL-1β and TNF-α and SMAD pathway activator Myostatin. The ability of each compound to block the atrophy induced by these different atrophy inducers was determined.

All three steroids can inhibit the atrophy induced by the inflammatory cytokines and Myostatin.

TABLE 5
Results of atrophy rescue assays

| Compounds | IL-1 | TNF-alpha | Myostatin |
|---|---|---|---|
| Corticosterone | ++ | ++ | +++ |
| Nestorone | ++ | + | ++ |
| Mifepristone | +++ | ++ | +++ |
| IGF-1 | +++ | +++ | +++ |

(+++: total rescue, ++: partial rescue, + low rescue)

In Vivo Assay

The benefits of Mifepristone on skeletal muscle function of a mouse model of Duchenne muscular dystrophy, the DMD mdx mouse, is evaluated.

After 6 weeks of treatment at 10 or 40 mg/day/kg an analysis of muscle function is performed 1) in vivo, by carrying out the grip test and wire test, 2) in situ by the analysis of contractile properties of isolated muscle and 3) in vitro, by analyzing the contractile properties of isolated permeabilized fibers from the diaphragm, a muscle described as one of the most affected muscles in the DMD mdx mouse. These approaches are completed by an analysis of plasma creatine kinase levels (CK), an indicator of muscle injury.

1-47. (canceled)

48. A method of treating a disease or disorder resulting in loss of skeletal muscle tissue and/or muscle weakness comprising administering to a patient in need of treatment a compound of formula (I) 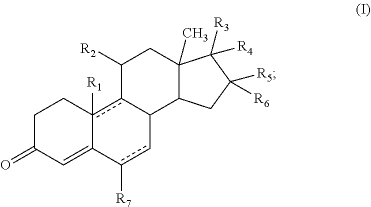
wherein:
R₁ is hydrogen or a C₁-C₃ alkyl, or is absent;
R₂ is selected from the group consisting of hydrogen, a hydroxyl group, a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;
R₃ is a hydroxyl group, —C(═O)R₈ with R₈ being a C₁-C₃ alkyl optionally substituted by a hydroxyl group, or —O—C(═O)R₁₁ with R₁₁ being a C₁-C₆ alkyl group optionally substituted by a carboxyl group;
R₄ is hydrogen, an acetoxy group or a C₁-C₆ alkyl, C₂-C₆ alkenyl or C₂-C₆ alkynyl group optionally substituted by a hydroxyl group or a halogen; or
R₃ and R₄ taken together form a tetrahydrofuran group optionally substituted by a methylene group;
R₅ and R₆ are hydrogen or taken together form a methylene group;
R₇ is hydrogen or methyl;
a and b respectively denote, independently from each other, a single bond or a double bond,
with the proviso that R₁ is absent when a is a double bond; with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group;
or any pharmaceutically acceptable diastereoisomer, salt, hydrate, solvate or prodrug thereof.

49. The method according to claim 48, wherein formula (I) is

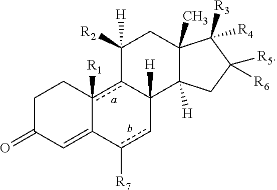

50. The method according to claim 48, wherein the compound of formula (I):
wherein:
R₁ is hydrogen or a C₁-C₃ alkyl, or is absent;
R₂ is selected from the group consisting of hydrogen, a hydroxyl group, a 4-dimethylamino-phenyl group, a 4-methylamino-phenyl group and an aminophenyl group;
R₃ is a hydroxyl group or —C(═O)R₈ with R₈ being a C₁-C₃ alkyl or —CH₂OH;
R₄ is hydrogen, an acetoxy group or a propynyl group;
R₅ and R₆ are hydrogen or taken together form a methylene group;
R₇ is hydrogen or methyl;
a and b respectively denote, independently from each other, a single bond or a double bond, with the proviso that R₁ is absent when a is a double bond;
with the provisos that (i) R₄ is an acetoxy group or a propynyl group and R₁ is a C₁-C₃ alkyl when R₇ is methyl, (ii) R₂ is selected from the group consisting of a hydroxyl group, a 4-dimethylamino-phenyl group, a 4-methylamino-phenyl group and an aminophenyl group when R₃ is —C(═O)R₈ with R₈ being —CH₂OH and (iii) R₄ is an acetoxy group or a propynyl group when R₃ is a hydroxyl group;
or any pharmaceutically acceptable, salt, hydrate, solvate or prodrug thereof.

51. The method according to claim 48, wherein:
R₁ is hydrogen or methyl, or is absent;
R₂ is selected from the group consisting of hydrogen, a hydroxyl group and a 4-dimethylamino-phenyl group;
R₃ is a hydroxyl group or —C(═O)R₈ with R₈ being methyl or —CH₂OH;
R₄ is hydrogen, an acetoxy group or a 1-propynyl group; and
R₇ is hydrogen or methyl.

52. The method according to claim 48, wherein R₁ is hydrogen or methyl.

53. The method according to claim 48, wherein R₂ is hydrogen or a hydroxyl group.

54. The method according to claim 48, wherein R₃ is —C(═O)R₈ with R₈ being methyl or —CH₂OH.

55. The method according to claim 48, wherein R₄ is hydrogen or an acetoxy group.

56. The method according to claim 48, wherein R₁ is methyl.

57. The method according to claim 48, wherein R₄ is an acetoxy group.

58. The method according to claim 48, wherein:
R₁ is absent;
R₂ is a phenyl group substituted, preferably in para position, by a dimethylamino group, a methylamino group, an amino group, a C₁-C₃ thioalkyl group, a dimethylamino N-oxide group, or —C(═O)R₁₀ with R₁₀ being a C₁-C₃ alkyl optionally substituted by a hydroxyl group;
R₃ is a hydroxyl group or —O—C(═O)R₁₁ with R₁₁ being a C₁-C₆ alkyl group optionally substituted by a carboxyl group;
R₄ is a C₁-C₆ alkyl, C₂-C₆ alkenyl or C₂-C₆ alkynyl group optionally substituted by a hydroxyl group or a halogen; or
R₃ and R₄ taken together form tetrahydrofuran group optionally substituted by a methylene group;
R₅ and R₆ are hydrogen;
R₇ is hydrogen.

59. The method according to claim 58, wherein R₂ is a phenyl group substituted, preferably in para position, by a dimethylamino group, a methylamino group, an amino group, a thiomethyl group, a dimethylamino N-oxide group, or —C(═O)R₁₀ with R₁₀ being a methyl.

60. The method according to claim 58, wherein R₃ is a hydroxyl group or —O—C(═O)R₁₁ with R₁₁ being an ethyl group optionally substituted by a carboxyl group.

61. The method according to claim 58, wherein R₄ is a C₂-C₃ alkyl group, C₂-C₃ alkenyl or C₂-C₃ alkynyl group optionally substituted by a hydroxyl group or a halogen, preferably chlorine.

62. The method according to claim 61, wherein R₄ is a propynyl group optionally substituted by a hydroxyl group.

63. The method according to claim 48, wherein said compound is selected from the group consisting of:

| Name | Formula |
|---|---|
| Mifepristone | 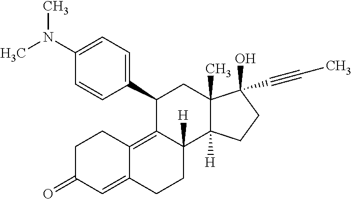 |
| Corticosterone | 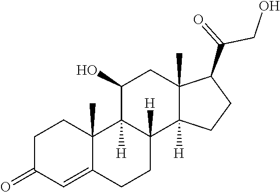 |
| Progesterone | 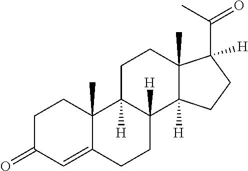 |
| Melengestrol acetate | 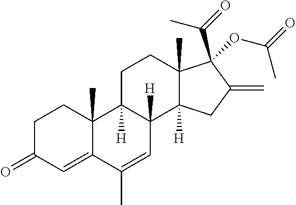 |
| Megestrol acetate | 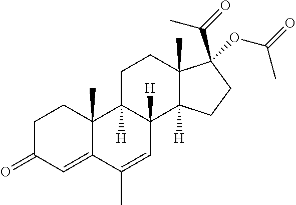 |
| Nestorone | 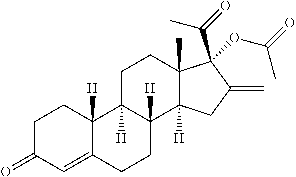 |
| RU42633 | 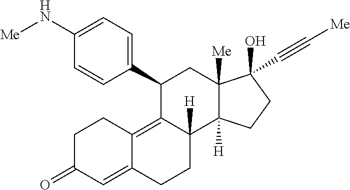 |
| RU42848 | 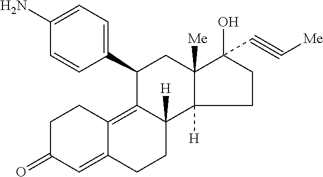 |
| RU42698 | 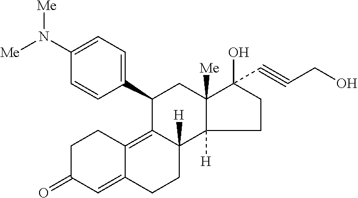 |
| Lilopristone | 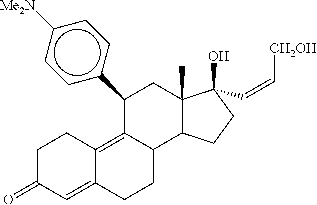 |
| Onapristone | 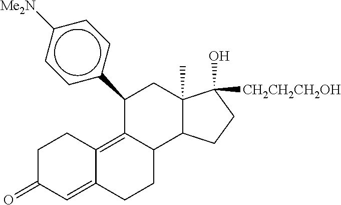 |
| ORG 31710 | 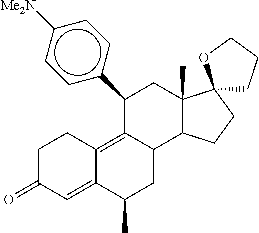 |
| ORG 33628 | 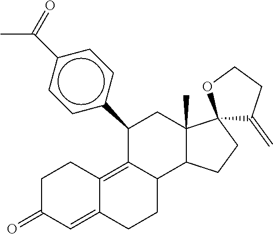 |
| RU 46556 | 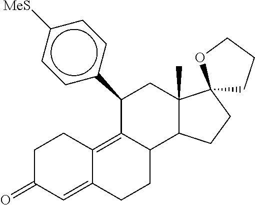 |
| RU 39973 | 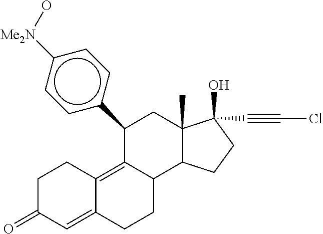 |
| RU 52562 | 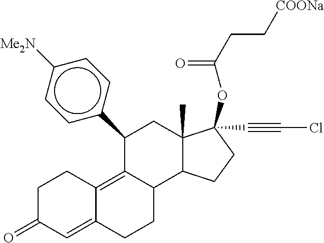 |
| Aglepristone | 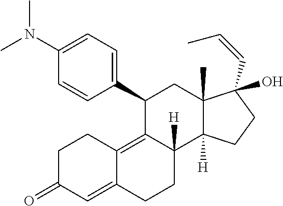 |

or any pharmaceutically acceptable diastereoisomer, salt, hydrate, solvate or prodrug thereof.

64. The method according to claim 48, wherein the disease or disorder resulting in loss of skeletal muscle tissue and/or muscle weakness, is selected from sarcopenia, cachexia, neuromuscular diseases, a muscular dystrophy, muscle disuse atrophy, atrophy induced by anorexia food starvation, muscle injuries, acute muscular injury or muscle overuse injury.

65. A method of inducing skeletal muscle hypertrophy and/or promoting skeletal muscle regeneration and/or reducing skeletal muscle atrophy comprising administering a compound as defined in claim 48 to a patient in need of treatment.

66. The method according to claim 65, wherein the disease or disorder resulting in loss of skeletal muscle tissue and/or muscle weakness is a muscular dystrophy.

67. The method according to claim 66, wherein the disease or disorder resulting in loss of skeletal muscle tissue and/or muscle weakness is Duchenne muscular dystrophy.

68. A product containing a compound of formula (I) as defined in claim 48, and a compound inducing skeletal muscular atrophy, as a combined preparation for simultaneous, separate or sequential use.

69. The product according to claim 68, wherein the compound inducing skeletal muscular atrophy is a therapeutic agent selected from the group consisting of corticosteroids, colchicine, chloroquine, hydroxychloroquine, D-penicillamine, antibiotics, betablockers, amiodarone, cimetidine, zidovudine, vincristine, clofibrate, statins, fibrates, cyclosporine, L-tryptophan, drugs causing hypokalaemia, lipid lowering agents, and therapeutic agents administered by intramuscular route.

70. A method to increase muscle mass, muscle strength and/or muscle performance in a subject comprising the administration of a compound of formula (I) as defined in claim 48 to said subject.

71. A method to reduce loss of skeletal muscle mass in a subject comprising the administration of a compound of formula (I) as defined in claim 48 to said subject.

72. An animal feed composition comprising a compound of formula (I) as defined in claim 48 and animal feed.

73. A method of improving livestock performance comprising providing to said livestock a compound of formula (I) as defined in claim 48.
